(19) United States
(12) Patent Application Publication
(10) Pub. No.: US 2024/0359163 A1
(43) Pub. Date: Oct. 31, 2024

(54) CATALYST SUPPORT STRUCTURES AND METHODS

(71) Applicant: UNIVERSITY OF CAPE TOWN, Rondebosch, Cape Town (ZA)

(72) Inventors: Michael Christian Maximilian CLAEYS, Cape Town (ZA); Nico Frederik FISCHER, Cape Town (ZA)

(73) Assignee: UNIVERSITY OF CAPE TOWN, Rondebosch, Cape Town (ZA)

(21) Appl. No.: 18/571,839

(22) PCT Filed: Jun. 29, 2022
(86) PCT No.: PCT/IB2022/056062

§ 371 Date: Dec. 19, 2023

(30) Foreign Application Priority Data
Jun. 30, 2021 (GB) 2109417.2

(51) Int. Cl. B01J 21/04; B01J 23/00; B01J 35/70; B01J 37/03

(52) U.S. Cl. CPC B01J 21/04; B01J 23/002; B01J 35/733; B01J 37/035; B01J 2231/648

(57) ABSTRACT

A method of preparing a catalyst support structure for use in a catalytic reaction. According to the method, a mixed metal oxide compound which defines a crystal lattice is synthesized. Cations of at least one catalytic promoter element are dispersed within the compound and incorporated into the crystal lattice. The conditions of synthesis are preselected to inhibit destabilization of the catalyst support structure such that the structure remains stable against collapse and exsolution under reaction conditions associated with the catalytic reaction. The metal oxide compound may comprise an oxidic perovskite having the formula A(1-x)A′(x)B(1-y)B′yO₃ wherein A and B represent metal cations and A′ and B′ represent cations of the promoter element or elements. Also provided is a catalyst support structure having cations of a promoter element incorporated into its crystal lattice. The support structure is stable against collapse and exsolution under reaction conditions.

### CROSS-REFERENCE TO RELATED APPLICATION

This application claims priority from United Kingdom patent application number 2109417.2 filed on 30 Jun. 2021, which is incorporated by reference herein.

### FIELD OF THE INVENTION

This invention relates to catalyst support structures for catalytic reactions involving the use of promoters, and to methods for their preparation and use in catalysis.

### BACKGROUND TO THE INVENTION

Catalysts are important for increasing the rate of reactions whilst remaining unchanged themselves. In heterogeneous catalysis the phase of the catalyst differs from that of the reactants. Heterogenous catalysis is important as it enables efficient, large scale production of end products. Important industrial examples which rely on this type of catalysis are the Haber-Bosch process, Fischer-Tropsch synthesis (FTS), steam reforming, olefin polymerization and the sulfuric acid synthesis, to name a few.

Heterogeneous catalysts are most often provided as supported catalysts, with catalytic nanoparticles being anchored to high surface-area area inorganic support materials which act as physical carriers. These carriers are referred to as catalyst support structures or, more simply, catalyst supports. Their high surface areas help to keep the active phase crystallites separated, suppressing crystallite growth (sintering) and the associated loss in active surface area. While the support itself is not usually catalytically active, it often plays a crucial role in modulating the redox chemistry and stability of the active site. Furthermore, catalyst supports play an important role in tailoring the local chemical environment surrounding the active site during catalysis through the adsorption of reactants and intermediates.

Promoters are substances which are used in conjunction with catalysts to improve their performance. Promoters are conventionally added to the surface of a catalyst through impregnation or precipitation. Promoters may influence catalytic activity, selectivity and/or stability. They may, for example, lower the activation energy required by the catalyst. Some promoters interact with active components of catalysts and thereby alter their electronic structure. Widely applied promoters can be found amongst the alkali and alkaline earth metals as well as the first two rows of the transition metals. By themselves, promoters typically have little or no catalytic effect in the reaction of choice.

As mentioned, one of the standard methods of using promoters involves impregnating the catalyst and promoter onto the support structure. For example, an iron-based catalyst precursor can be prepared by impregnating an aqueous Fe(NO₃)·9H₂O solution onto an SiO₂ support. After impregnation of the Fe, a desired amount of K is added by aqueous impregnation with K₂CO₃, followed by aqueous impregnation with CuNO₃. The precursors are then calcined to obtain the final supported catalysts. Such impregnation techniques have various drawbacks, however.

As a result of generally low concentrations of the promoters in catalyst formulations, their exact location and speciation, especially under reaction conditions, is often not fully understood. In addition, under reaction conditions promoters have been reported to be metastable, changing phase and even moving on the catalyst surface. These effects can cause a depletion of the promoter in some regions of the catalyst, or enrichment of the promoter beyond desired levels in others. Both effects may result in a deterioration of the catalyst or its activity.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with an aspect of the invention there is provided a method of preparing a catalyst support structure for use in a catalytic reaction, the method comprising the steps of
synthesizing a mixed metal oxide compound having a crystallographic phase which defines a crystal lattice, the compound being configured to support a catalyst for a catalytic reaction, the compound further having a group of cations of at least one catalytic promoter element being dispersed within the compound and incorporated into the crystal lattice, and the promoter element being capable of promoting the catalytic reaction;
wherein the conditions of the synthesis are preselected to inhibit destabilization of the catalyst support structure such that the structure remains stable against collapse and exsolution under reaction conditions associated with the catalytic reaction.

The conditions of the synthesis of the metal oxide compound may be preselected to provide that the resultant metal oxide compound comprises a perovskite structure, a hydrotalcite structure, or a structure derived from either one of these.

In one mode of performing the method, the conditions of the synthesis are preselected such that the metal oxide compound is synthesized as an oxidic perovskite. The oxidic perovskite may have the following formula CHEM 1:

A₍₁₋ₓ₎A′₍ₓ₎B_{(1-y)}B′_{y}O₃ CHEM 1:

wherein:
A and B represent metal cations having ionic radii R_{A} and R_{B} respectively;
A′ and B′ represent cations of at least one promoter element;
O represents an oxygen atom having an ionic radius R_{O}; and
R_{A}+R_{O}=t×sqrt(R_{B}+R_{O}), wherein t is close to unity.

Without limitation thereto, the metal cation A may be a cation of La or Bi. Also without limitation, the metal cation B may be a cation of an element selected from the group consisting of Al, Ti, Zn and Mo.

The parameter “t” may have a value ranging from about 0.7 to about 1.3. It may have a value of about 1±0.1.

The conditions of the synthesis of the metal oxide compound may be preselected such that the promoter element (or elements) are added to the synthesis in an appropriate stoichiometry to produce the structure of formula CHEM 1.

The conditions of the synthesis of the metal oxide compound may be preselected so that the resulting metal oxide compound lacks regions likely to exsolute under conditions associated with the catalytic reaction.

The promoter element (or each promoter element if the embodiment incorporates co-promoters) may be independently selected from the group consisting of alkali, alkaline earth, and transition metals of groups 3 to 7 and periods 4 to 5 of the periodic table, following the current IUPAC numbering scheme. The promoter element or elements may be selected from a narrower group consisting of Mn, Nb, Zr, V, Ti, Ca, Mg, and the alkali metals excluding Fr. The promoter element or elements may be resistant to reduction. For example, they may have properties which make them substantially irreducible in a gas environment containing a reductive agent such as, but not limited to, H₂ and CO up to a temperature of about 750° C.

Cations of a plurality of catalytic promoter elements may be incorporated into the crystal lattice, effective as co-promoters of the catalytic reaction. For example, the catalyst support structure may have K and Mn incorporated as co-promoters.

The method of preparing the support structure may include a step of depositing onto the mixed metal oxide compound (e.g., the perovskite) a catalyst capable of catalysing said catalytic reaction. The catalyst may be deposited as a catalytically active phase. The step of depositing the catalyst may be performed by at least one technique selected from the group consisting of deposition, impregnation, and precipitation.

The step of depositing the catalyst onto the mixed metal oxide compound may be performed subsequently to the step of synthesizing the mixed metal oxide compound.

It will be appreciated that the reaction conditions will vary depending upon the specific reaction to be catalysed. Without limiting the generality thereof, the reaction conditions may be selected from groups of conditions suitable for Fischer-Tropsch synthesis, Haber-Bosch processes, decomposition of nitrogen oxides (NOx) and N₂O, dry reforming of CO₂, steam reforming of methane, CO and CO₂ hydrogenation, (reverse) water gas shift reactions, soot oxidation, and the synthesis of higher alcohols, including synthesis over Cu based catalysts.

Correspondingly, the catalytic reaction for which the support structure is prepared may be one of those described above. However, it may instead comprise any one of a wide variety of other appropriate catalytic reactions. The disclosed method of preparing a catalyst support structure may thus find application in numerous industrial and chemical processes involving catalysis. The catalytic reaction may comprise a heterogeneous catalytic reaction.

The method of preparing the support structure may further include performing activation treatment on the catalytically active phase.

The invention extends to a catalyst support structure prepared using the method described above.

During use of the prepared catalyst support structures, the promoter cations may remain in the crystal lattice upon exposure to activation or reaction conditions associated with the catalyst, and the catalyst support structure may remain intact and exsolute substantially no components upon exposure to activation or reaction conditions. Accordingly, the disclosed method may be suitable for preparing a catalyst support structure that is stable against collapse under the conditions of the reaction being catalysed.

Moreover, it may be expected that the promoter elements incorporated in the support structure (e.g., the cations A′ and B′ in the general formula CHEM 1) will enhance the activity and selectivity of the catalytically active phase on the perovskite surface.

In accordance with a further aspect of the invention there is provided a catalyst support structure comprising
a mixed metal oxide compound having a crystallographic phase which defines a crystal lattice, the compound being configured to support a catalyst for a catalytic reaction; and
a group of cations of at least one catalytic promoter element dispersed within the compound and incorporated into the crystal lattice, the promoter element being capable of promoting the catalytic reaction;
wherein the catalyst support structure is stable against collapse and exsolution under reaction conditions associated with the catalytic reaction.

The mixed metal oxide compound may have a structure selected from the group consisting of perovskite structures, hydrotalcite structures, and structures derived therefrom.

In one embodiment, the mixed metal oxide compound may have a perovskite structure. It may comprise an oxidic perovskite. The oxidic perovskite may have the formula CHEM 1 as set out above.

The perovskite may comprise La₍₁₋ₓ₎KₓAl_{(1-y)}Mn_{y}O₃ wherein 0<x≤0.2 and y≤1. In certain embodiments, x≤0.1. Without limitation thereto, the perovskite may comprise a compound selected from the group consisting of La_{0.9}K_{0.1}AlO₃ and La_{0.9}K_{0.1}Al_{0.8}Mn_{0.2}O₃.

Further details of the disclosed catalyst support structure may be as set out above for the disclosed method of preparation. Corresponding embodiments may accordingly also be applicable for these aspects of the invention.

The invention extends to a catalyst support structure for use in heterogeneous catalysis of a chemical reaction, the structure comprising a perovskite with a crystal lattice defining a surface configured to support an active catalyst phase and having a plurality of atoms of at least one promoter element distributed generally uniformly across the surface and within the crystal lattice; wherein said promoter element is effective to promote catalysis of the chemical reaction.

Further details of this embodiment of the catalyst support structure may be as hereinbefore described, and corresponding embodiments may accordingly also be applicable for this aspect of the invention.

The invention extends further to an assembly comprising a catalytically active phase and a catalyst support structure onto which the catalytically active phase is loaded, the catalyst support structure being substantially as hereinbefore described; wherein the catalytically active phase is substantially free of cations of the promoter element.

Further details of the catalyst support structure may be as hereinbefore described.

In accordance with a further aspect of the invention there is provided a method of performing a catalytic reaction, the method comprising the steps of
supporting a catalyst on a catalyst support structure as described above;
subjecting the catalyst to activation treatment thereby to provide a catalytically active phase of the catalyst loaded on the catalyst support structure;
exposing the catalyst support structure with the loaded active phase to reaction conditions;
contacting at least one reactant with the loaded active phase; and
applying activation energy to the reactant, thereby to convert the reactant to at least one product.

Further details of the catalyst support structure used in the method may be as hereinbefore described. It may comprise a perovskite structure having the general formula CHEM 1. Corresponding embodiments may accordingly also be applicable for these aspects of the invention.

The catalytic reaction and the reaction conditions associated with it may be as described above. Corresponding modes may accordingly also be applicable for these aspects of the invention.

Embodiments and modes of performing the invention will now be described, by way of example only, with reference to the accompanying drawings.

### BRIEF DESCRIPTION OF THE DRAWINGS

In the drawings:

FIG. 1 is a series of energy dispersive spectroscopy (EDS) maps of La, K and Mn in La_{0.9}K_{0.1}Al_{0.8}Mn_{0.2}O₃, derived from transmission electron microscopy (TEM) studies;

FIG. 2 are X-ray diffraction patterns of La₁₋ₓKₓAl_{1-y}Mn_{y}O₃ with x=0 and 0.1 and y=0, 0.2, 0.6 and 1 displaying only reflexes of the perovskite structure;

FIG. 3 is a schematic three-dimensional diagram of a slab model generated for a catalyst support structure as disclosed herein, comprising La_{0.9}K_{0.1}AlO₃;

FIG. 4 is a graph illustrating CO conversion obtained during Fischer-Tropsch synthesis (FTS) using Fe supported on a variety of different perovskite materials, after 40 to 48 hours of Time on Stream (TOS) at T=240° C., P=15 bar, H₂/CO=2, Space Velocity (SV)=8 ml·min⁻¹·g⁻¹;

FIG. 5 is a graph illustrating CO conversion obtained during FTS using Fe supported on a variety of different perovskite materials, after 40 to 48 hours of TOS at T=240° C., P=15 bar, H₂/CO=2, SV=8 ml·min¹·g⁻¹ (but for the samples with an HSV suffix, where SV=30 ml·min⁻¹·g⁻¹);

FIG. 6 is a graph illustrating CO₂ selectivity obtained during FTS using Fe supported on a variety of different perovskite materials, after 40 to 48 hours of TOS at T=240° C., P=15 bar, H₂/CO=2, SV=8 ml·min⁻¹·g⁻¹ (but for the samples with an HSV suffix, where SV=30 ml·min⁻¹·g⁻¹);

FIG. 7 is a graph illustrating methane selectivity, as fraction of the hydrocarbon product, obtained during FTS using Fe supported on a variety of different perovskite materials, after 40 to 48 hours of TOS at T=240° C., P=15 bar, H₂/CO=2, SV=8 ml·min¹·g⁻¹ (but for the samples with an HSV suffix, where SV=30 ml·min⁻¹·g⁻¹);

FIG. 8 is a graph illustrating C5+ selectivity, as fraction of the hydrocarbon product, obtained during FTS using Fe supported on a variety of different perovskite materials, after 40 to 48 hours of TOS at T=240° C., P=15 bar, H₂/CO=2, SV=8 ml·min¹·g⁻¹ (but for the samples with an HSV suffix, where SV=30 ml·min⁻¹·g⁻¹);

FIG. 9 is a graph illustrating the olefin to paraffin ratio in the C5 fraction obtained during FTS using Fe supported on a variety of different perovskite materials, after 40 to 48 hours of TOS at T=240° C., P=15 bar, H₂/CO=2, SV=8 ml·min⁻¹·g⁻¹ (but for the samples with an HSV suffix, where SV=30 ml·min⁻¹·g⁻¹);

FIG. 10 is a graph illustrating CO conversion obtained during FTS using Fe supported on two different perovskite materials prepared using PMMA spheres as templates, after 40 to 48 hours of TOS at T=240° C., P=15 bar, H₂/CO=2, SV=8 ml·min⁻¹·g⁻¹;

FIG. 11 is a graph illustrating CO₂ selectivity obtained during FTS using Fe supported on two different perovskite materials prepared using PMMA spheres as templates, after 40 to 48 hours of TOS at T=240° C., P=15 bar, H₂/CO=2, SV=8 ml·min⁻¹·g⁻¹;

FIG. 12 is a graph illustrating methane selectivity, as fraction of the hydrocarbon product, obtained during FTS using Fe supported on two different perovskite materials prepared using PMMA spheres as templates, after 40 to 48 hours of TOS at T=240° C., P=15 bar, H₂/CO=2, SV=8 ml·min⁻¹·g⁻¹;

FIG. 13 is a graph illustrating C5+ selectivity, as fraction of the hydrocarbon product, obtained during FTS using Fe supported on two different perovskite materials prepared using PMMA spheres as templates, after 40 to 48 hours of TOS at T=240° C., P=15 bar, H₂/CO=2, SV=8 ml·min⁻¹·g⁻¹;

FIG. 14 is a graph illustrating an X-ray absorption spectrum of the manganese K edge of La_{0.9}K_{0.1}Al_{0.4}Mn_{0.6}O₃ as prepared and after reductive treatment in hydrogen at 450° C.; and

FIG. 15 is a graph illustrating an X-ray absorption spectrum of the potassium K edge of La_{0.9}K_{0.1}Al_{0.4}Mn_{0.6}O₃ as prepared and after reductive treatment in hydrogen at 450° C.

### DETAILED DESCRIPTION WITH REFERENCE TO THE DRAWINGS

Embodiments of the disclosed catalyst support structures and methods for their preparation and use are explained in greater detail in the following description.

During catalytic reactions the active phase commonly needs to be reduced or carburized. If present in the support structure, this is typically achieved by exsolution. This is the focus of many previous disclosures in this field. The general approach was to provide perovskites that are unstable and which would potentially be destroyed during the catalyst formation process.

In contrast to such previous techniques, the embodiments of the present invention are synthesized in such a manner that destabilization is inhibited or prevented. The presently disclosed support structures are not intended to be destroyed. A stable structure is created rather than a destabilized structure. The design of the present structures is such that the cations of the promoter element (or elements) are incorporated into the crystal lattice of the support structure and not into the catalytically active phase. The promoter cations are thus atomically distributed in the crystal lattice and may be locked or positionally anchored with respect to adjacent atoms in the lattice. As a result, they generally do not exsolute from the disclosed catalyst support structures under activation or reaction conditions. The present support structures are therefore stable against collapse and exsolution under activation and reaction conditions associated with the catalytic reactions for which they are used. They are typically more difficult to reduce than past support structures, from which the catalytically active phase needed to be exsoluted during use.

Aspects of the invention provide a method of preparing a catalyst support structure for use in a catalytic reaction. The method may include synthesizing a mixed metal oxide compound having a crystallographic phase which defines a crystal lattice. The compound is typically configured to support a catalyst for a catalytic reaction. Cations of one or more catalytic promoter elements are dispersed within the compound and incorporated into its crystal lattice. The reaction conditions of the synthesis are preselected with a view to inhibiting, impeding, blocking or otherwise preventing destabilization of the catalyst support structure. This may be done by synthesizing the metal oxide compound so that it lacks regions likely to exsolute under conditions associated with the catalytic reaction. The result is that the structure remains stable against collapse and exsolution under reaction conditions.

The conditions of the synthesis of the mixed metal oxide compound may be preselected to provide an oxidic perovskite. The perovskite may have the formula CHEM 1 as set out in the summary of the invention above. The variables and parameters of the formula CHEM 1 may also be as set out in the summary.

As described in the summary, the catalyst support structure may comprise a combination of different promoter elements effective as co-promoters of the catalytic reaction.

It will be appreciated that the promoter element (or elements when co-promoters are provided) may be preselected based on their capability to promote or co-promote the catalytic reaction of interest. Elements may therefore be chosen which have properties favourable for promoting the catalytic reaction which the supported catalyst is effective to catalyse. The promoter elements should therefore be capable of promoting or co-promoting the catalytic reaction for which the catalyst and its support structure are intended to be used.

For embodiments in which the metal oxide has a perovskite structure, the promoter elements in cationic form may be incorporated in the crystal lattice of the perovskite structure and are typically stable under the chosen reaction conditions. The cations of the promoter element (or elements) may be incorporated into the crystal lattice by ionic bonding to adjacent atoms of the lattice. The cations may be locked or positionally anchored in the crystal lattice relatively to said adjacent atoms.

It will be appreciated that the disclosed support structures will be suitable for a wide variety of different catalytic reactions, and that the reaction conditions will vary depending on the specific reaction being catalysed. Some non-limiting examples of suitable reaction conditions have been described in the summary of the invention above. The catalytic reaction may be a heterogeneous catalytic reaction.

The following are presented as independent, non-limiting examples of reaction conditions for which the disclosed catalyst support structure may be suitable:
Fischer-Tropsch conditions in a slurry reactor at 240° C., 15 bar and a H₂ to CO ratio of 2;
Reductive treatment in hydrogen at approximately 450° C.; or
Reducing conditions at temperatures in excess of 450° C.

In certain embodiments, the catalyst may be iron-based.

The method of preparing the support structure may further include depositing a catalyst onto the prepared metal oxide compound, e.g., the perovskite. The catalyst may be preselected to be suitable to catalyse the required reaction. The catalyst may be deposited as a catalytically active phase.

The step of depositing the catalyst onto the metal oxide compound may be performed by a technique selected from the group consisting of deposition, impregnation, and precipitation.

The step of depositing the catalyst onto the metal oxide compound may be performed subsequently to the synthesis step. For example, where the mixed metal oxide compound is a perovskite, the step of depositing the catalyst onto the perovskite may be performed as a subsequent step to the perovskite synthesis step, that is, after the perovskite with its incorporated promoter elements has been synthesized. This may distinguish the present method over previously described methods of preparing support structures. Instead of adding the catalytically active phase while synthesizing the perovskite structure (as in previous methods), this step can be omitted during the synthesis and left until later. Stated differently, the steps which are used to make the present support structure can be performed in a different order than the order of previous preparation steps, since in the present method the addition of the catalytically active phase can be completed after synthesis of the perovskite structure into which the promoter elements are incorporated. Surprising advantages may result from omitting the catalyst addition step during the synthesis of the perovskite. For example, exsolution of the promoter elements under reaction conditions may be substantially avoided and collapse of the support structure is therefore inhibited. The incorporation of the promoter cations A′ and B′ into the oxidic perovskite before deposition of the catalyst (and before activation treatment) means that the promoter elements do not reduce and exsolute from the perovskite structure during activation treatment of the catalytically active phase.

The method of preparing the catalyst support structure may further include performing activation treatment on the catalytically active phase.

It will be appreciated that the promoter element (or elements) must be added to the synthesis of the perovskite in an appropriate stoichiometry to produce the structure of formula CHEM 1.

During use of the disclosed support structure, the majority of the promoter cations remain in the crystal lattice upon exposure to activation or reaction conditions associated with the catalyst, and the support structure remains substantially intact. Exsolution of components of the structure upon exposure to activation or reaction conditions is negligible for practical purposes. Accordingly, the disclosed method may be suitable for preparing a catalyst support structure that is stable against collapse under reaction conditions associated with the catalysed reaction.

In addition to the method described above, a catalyst support structure is also disclosed. This comprises a mixed metal oxide compound having a crystallographic phase which defines a crystal lattice. The compound is typically configured to support a catalyst for a catalytic reaction. Cations of at least one catalytic promoter element are dispersed within the compound and incorporated into the crystal lattice. The promoter element are selected to be capable of promoting the catalytic reaction. The catalyst support structure is stable against collapse and exsolution under reaction conditions associated with the catalytic reaction.

The mixed metal oxide compound may have a structure selected from the group consisting of perovskite structures, hydrotalcite structures, and structures derived therefrom.

The mixed metal oxide compound may comprise an oxidic perovskite. It may have the the formula CHEM 1 as set out in the summary of the invention above.

Without limitating the generality of suitable compounds, oxidic perovskites that may be suitable for the presently disclosed catalyst support structures may have the general formula La₍₁₋ₓ₎KₓAl_{(1-y)}Mn_{y}O₃ wherein 0<x≤0.2 and y≤1. In certain embodiments, x may be ≤0.1. For example, and without limitation thereto, the perovskite may comprise La_{0.9}K_{0.1}AlO₃ or La_{0.9}K_{0.1}Al_{0.8}Mn_{0.2}O₃.

Other details of the catalyst support structure may be as described above in reference to the method of preparing the support structure, and corresponding embodiments may accordingly also be applicable for these aspects of the invention.

The invention extends to a catalyst support structure comprising a mixed metal oxide having a perovskite structure configured to support a catalyst, with a group of cations of one or more promoter elements dispersed within a crystallographic phase of the perovskite support structure; wherein the cations are incorporated into the crystallographic phase and remain in it upon exposure to activation or reaction conditions associated with the catalyst.

The invention also extends to a catalyst support structure for use in heterogeneous catalysis of a chemical reaction, the structure comprising a perovskite which includes at least two metal elements and which has a crystal lattice, and cations of at least one promoter element incorporated into the crystal lattice; wherein cations of said promoter element replace atoms of at least one of the metal elements in the crystal lattice; and wherein the promoter element (or each element when there are more than one) is effective to promote catalysis of the relevant chemical reaction.

The invention also extends to a catalyst support structure for use in heterogeneous catalysis of a chemical reaction, the catalyst support structure comprising a perovskite structure with a crystal lattice defining a surface configured to support an active catalyst phase and having a plurality of atoms of at least one promoter element distributed generally uniformly across the surface and within the crystal lattice; wherein the promoter element (or each element when there are more than one) is effective to promote catalysis of the chemical reaction.

Also provided are perovskite structures onto which a metal or metal oxide can be deposited. The metal or metal oxide is selected to be capable of forming an active phase of a catalyst under activation or reaction conditions. The perovskite structure has a crystallographic phase into which at least one promoter element is incorporated. The promoter element (or elements) may be capable of enhancing activation of the deposited metal or metal oxide under reaction conditions without leaving the crystallographic phase. Instead, or in addition, the promoter element (or elements) may be capable of enhancing selectivity of the deposited metal or metal oxide under reaction conditions without leaving the crystallographic phase. In both cases, the perovskite may be an oxidic perovskite. The oxidic perovskite may have the general structure CHEM 1 as described above. The metal cations and promoter element (or elements) may be as described above.

Further details of the perovskite structures may be as hereinbefore described with reference to the disclosed catalyst support structures. Corresponding embodiments may accordingly also be applicable for these aspects of the invention.

A catalytic assembly for use in a catalytic reaction is also provided. The assembly may comprise a catalytically active phase and a catalyst support structure onto which the catalytically active phase is loaded. The support structure may be as described in this specification. It may therefore comprise a mixed metal oxide compound having a crystal lattice and a plurality of cations of at least one promoter element incorporated into the lattice. The catalytically active phase may be substantially free of cations of the promoter element.

A method of performing a catalytic reaction using the disclosed catalyst support structures is also disclosed. The method may involve supporting a catalyst on an embodiment of a catalyst support structure as described above and subjecting the catalyst to activation treatment so that a catalytically active phase of the catalyst is loaded on the catalyst support structure. The catalyst support structure with the loaded active phase may be exposed to reaction conditions. One or more reactants may be contacted with the loaded active phase. Activation energy may be applied to the system including the reactant, thereby to convert the reactant to at least one product.

Further details of the catalyst support structure may be as hereinbefore described. It may have a perovskite structure with the general formula CHEM 1. Corresponding embodiments may accordingly also be applicable for these aspects of the invention.

The catalytic reaction and reaction conditions may be as described above in reference to the catalyst support structure.

The method of performing the catalytic reaction may involve combining one or more chemical reactants with a catalytic assembly as described above and applying activation energy to the combination to convert the reactant or reactants into one or more products.

It may be expected that the promoter elements incorporated in the support structure (e.g., the cations A′ and B′ in the perovskite structure having formula CHEM 1) will enhance the activity and/or selectivity of the catalytically active phase on the perovskite surface.

Also disclosed is a method of preparing a catalytic assembly for catalysing a catalytic reaction. The method comprises loading a catalyst on a catalyst support structure as described above. The catalyst is selected to be effective to catalyse the relevant catalytic reaction.

As discussed, the catalyst support structures may have crystalline perovskite structures. Perovskites have the general formula ABX₃ where A and B typically represent cations and X is an anion that bonds to both. The materials class of perovskite offers a very large scope for material design. For both A-site and B-site cations, a great variety of choices is conceivable. Additionally, for either lattice site, a combination of two or more (in principal with no restrictions) cations instead of a single element can be utilized to further tune the properties of a material. The possibilities range from doping a main component with only small amounts of another element to combining equal parts of several elements.

Furthermore, sub-stoichiometric perovskites exist, where either A-sites or B-sites are not fully occupied. All this makes the materials class highly versatile, as all incorporated elements contribute to the properties of the perovskite. Promoters may be incorporated into the A and B sites of the perovskite structure.

Although the component X in the general perovskite formula can be any suitable element, in some embodiments of the disclosed catalyst support structures it may be oxygen. Thus, oxidic perovskites are one example of a suitable structural framework for the disclosed catalyst support structures.

Perovskites are not commonly employed as heterogeneous catalysts. They have not been considered to be useful for providing suitable and stable catalyst support structures such as those discussed herein. Some academic studies have utilized the flexibility of their composition to design catalyst precursors. However, previous studies relating to perovskites have typically been concerned with providing a catalyst through exsolution from the perovskites. This has typically been done as an academic exercise only, without widespread application and not scaled to industrial use.

For example, iron or cobalt have been exsoluted from a perovskite structure in a reducing environment to yield the active phase. Perovskite oxides can be doped on the B-site with catalytically active elements. The perovskite host lattice then acts as a reservoir that can release these dopants upon reductive treatment or in reducing reaction environments. The crystal structure of the perovskite lattice is influenced, rendering the material less stable against reduction. During exsolution, dopants migrate to the surface of the perovskite structure where they form nanoparticles, thus creating active catalyst surfaces, often collapsing the host structure in the process. Perovskites that exhibit exsolution typically incorporate an easily reducible metal (which is then selectively reduced and exsolved). Examples of catalytic metals that have been used are Fe, Co, Ni, Pd, Rh, Ru and Pt.

In Goldwasser (2003), Fe was incorporated into a perovskite at a very high concentration as it was expected to exsolute under reductive treatment. The resultant compounds have 80-95% of the B site composed of Fe cations, and exsolution destroys the perovskite structure. Perovskites of that type may serve as a vehicle to provide the precursor of the active phase but not the structure present under reaction conditions.

In the present disclosure, cations of the promoter element or elements may be incorporated into the structure of the perovskite (the structural framework) so that they take the places commonly occupied by cations of the perovskite. It will be appreciated that this incorporation may occur during synthesis of the structural framework. Thus, the cations of the promoter element may be locked into the structural framework during synthesis of the framework. The formation of the structural framework and the incorporation of the promoter cations within the structural framework may be effected simultaneously as part of the overall synthetic process during manufacture of the catalyst support structure.

As discussed, the catalyst support structures may be configured to provide co-promoting functionalities. Thus, the structures may include cations of a combination of different promoter elements which can work together and are effective as co-promoters of the catalytic reaction in question. By way of example, in certain embodiments the catalyst support structure may comprise the promoter elements K and Mn which, in use, may operate cooperatively as co-promoters.

The promoter element or elements may be preselected for their capability to promote or co-promote the catalytic reaction for which the catalyst support structure is intended to be used. Thus, a given catalyst support structure may be designed and manufactured for use in a selected catalytic reaction. To this end, the promoter element or elements that are incorporated into the crystal lattice of the framework structure may be preselected prior to the manufacturing process, based on their predicted or empirically established effectiveness for promoting or co-promoting the catalytic reaction of interest.

Due to the incorporation of the promoter atoms into the framework structure, the resultant catalyst support structure may present a surface having a plurality of promoter cations distributed generally uniformly across it. The distribution of the promoter atoms may be generally more predictable than the distribution typically seen with conventional supports associated with promoters introduced via impregnation or precipitation. This is because the regular distribution of the promoter cations, and the fact that they are anchored or locked into the support structure, may mitigate against random agglomeration and sintering as well as mobility of the promoter elements.

It will be appreciated that the embodiments of the catalyst support structure disclosed in this specification may be combined with the active species of one or more catalysts, thereby providing catalytic assemblies which may be used to catalyse a given reaction.

There are a wide variety of ways to synthesize the above described catalyst support structures. In one mode presented here for illustrative purposes only, it may be performed by a sol-gel process. In another mode, the perovskite phase may be grown in the presence of a templating material, here poly(methyl methacrylate) (PMMA) microspheres, and the template may subsequently be removed by thermal or chemical treatment.

The described methods may yield a mixed metal support structure wherein cations of the promoter element are dispersed or interspersed through the mixed metal compound and are fixed in position relative to it.

### EXAMPLES

Embodiments of the disclosed catalyst support structures are described along with their use in iron-based Fischer-Tropsch synthesis (FTS). The FTS process is used merely as an example of a type of reaction for which the support structures may be useful, and many other reactions relying on catalyst promotion will benefit from their use.

The activity and selectivity of the tested support structures are illustrated in the accompanying Figures, with reference to the experiments described below.

Promotion in the FTS process can have advantageous effects on the reduction and formation of the catalyst as well as its performance. The most common promoters for FTS are K, Mn and Cu. Cu is generally accepted to act as a reduction promoter during the initial activation of the iron oxide catalyst precursor with some reports suggesting an improvement of the methane selectivity and a decrease in olefin content. Both K and Mn are reported to increase the adsorption energy of CO and CO₂ and to weaken the strength of the carbon-oxygen bond, thereby facilitating bond cleavage. This may support catalyst formation and activity and suppress hydrogenation and the formation of short chained paraffins.

Such mechanisms are not specific to the FTS process but have also been observed in other catalytic reactions such as the Haber-Bosch processes, the decomposition of nitrogen oxides (NOx) and N₂O, dry reforming of CO₂, steam reforming of methane, CO and CO₂ hydrogenation, (reverse) water gas shift reaction, soot oxidation, and the synthesis of higher alcohols, e.g., synthesis over Cu based catalysts.

In classical catalyst synthesis techniques, these promotion processes have an optimum as a function of promoter loading. At excessively high loadings of potassium for example, the catalyst begins to deteriorate as active sites are covered by carbonaceous or potassium species. Additionally, the mobility of promoters under reaction conditions results in a highly dynamic system which undergoes significant changes in performance during its lifetime. This is especially challenging when considering process intensification efforts to increase the overall process sustainability.

Perovskite materials were used to investigate the properties of the disclosed catalyst support structures. Various promoter elements were interspersed in the support structure.

Perovskite lanthanum aluminate (LaAlO₃) is an example of a mixed metal oxide substrate material which has unusual ferro-elastic properties and has been used previously as a support structure in conjunction with Fe as catalyst and K as a promoter. In classical techniques, the potassium promoter is not integrated within the structure of the lanthanum aluminate, however. The nomenclature Fe—K/LaAlO₃ is used in the descriptions which follow to connote such classical supports impregnated with potassium.

For present purposes, the elements K and Mn were selected as promoters to be used in the manufacture of the support structures to be tested. In the FTS process, K is understood to enhance CO conversion and shift the selectivity in favour of longer chains and away from methane. In the presence of both K and Mn, the CO conversion is also enhanced but to a lower extent. However, WGS activity is suppressed and the selectivity to short alkenes is significantly enhanced.

To provide a standard support material as a control for comparison, LaAlO₃ perovskite was prepared using a polymeric precursor method. A precursor solution was prepared by mixing equimolar amounts of metal salts of La(NO₃)₃·6H₂O and Al(NO₃)₃·9H₂O with citric acid, nitric acid, and deionised water. The concentration of citric and nitric acid was double and triple the total concentration of metal cations. The solution was ultra-sonicated to dissolve the metal salts. It was subsequently heated to 60° C. while stirring, at which point ethylene glycol was added in the molar ratio of 3:1 with citric acid. The solution was held at 90° C. for 1 hr to allow for polyesterification and then transferred to a hot mantle at 100° C. for another hour to dehydrate and form a gel. The resulting gel was heated to 350° C. in a hot mantle yielding a black powder. The precursor powder was well ground and calcined at 800° C. for 6 hours in static air to produce the perovskite compound.

The test materials were prepared individually from metal salt solutions according to the desired stoichiometry. A similar polymeric precursor method was used as that described above. Various perovskite materials as disclosed herein were prepared based on LaAlO₃. La was partially replaced with K and Al with Mn. Some of the materials produced for testing against the standard perovskite had the general formula La_{0.9}K_{0.1}Al_{(1-y)}Mn_{y}O₃. These materials were synthesized using equimolar amounts of metal cations on the A and B site of the perovskite structure. For K and Mn, the respective nitrate salts were used.

40 ml of each of a 0.25 M of FeCl₂·4H₂O, 5.4 M NaOH and 1.34 M NH₃·H₂O solutions were mixed. The mixture was stirred on a magnetic stirrer and heated up to 90° C. under an inert nitrogen atmosphere. The solution was kept at the temperature for 1.5 hours after which the pH was adjusted to 2.5 by dropwise addition of 40 ml of 0.1 M HCl solution. The slurry was then washed 20 times with boiling deionized water to remove residual chloride ions. The obtained iron oxide nanoparticles were resuspended in deionized water via ultrasonication and then mixed with the perovskite materials or reference supports (ZrO₂, SiO₂, Al₂O₃) to yield a loading of 16 wt.-% iron after filtration and drying.

In another mode of performing the invention, LaAlO₃ and La_{0.9}K_{0.1}AlO₃ were prepared in the presence of a hard template, namely poly(methyl methacrylate) (PMMA) microspheres. 10.0 g of LaNO₃·6H₂O and 8.66 g Al(NO₃)₃·9H₂O was dissolved in 5 ml ethylene glycol by slow stirring in a 100 ml beaker at room temperature. For the potassium containing sample, the appropriate amount of LaNO₃·6H₂O was replaced by potassium nitrate. The produced ethylene glycol solution was transferred into a 25 ml volumetric flask, to which 10 ml methanol and 10 ml ethylene glycol were added. The PMMA spheres were soaked in the solution and infiltration of the precursor solution into the particles was observed. The process took between 24 and 48 hours and was terminated once the colour of the spheres changed to the colour of the solution. The excess solution was separated from the impregnated PMMA colloidal crystals by vacuum filtration. The obtained samples were allowed to dry in air at room temperature followed by calcination at 800° C. for 5 hours in an air flow. To distinguish the materials prepared in the presence of a hard template the samples are termed La₁₋ₓKₓAlO₃—PMMA with x=0 and 0.1.

7.49 g Fe(NO₃)₃·9H₂O, was dissolved in 30 ml of deionized water and then added to the La₁₋ₓKₓAlO₃—PMMA perovskite support to obtain a loading of 16 wt.-% Fe. The incipient wetness was achieved by saturating the dry support with the metal precursor solution until a paste formed. The volume of solution required to form a paste was noted. The catalyst was then dried in a rotary evaporator at 80° C. and 150 mbars. The remaining precursor solution was topped with deionized water to the volume used in the first impregnation step and the second impregnation was carried out with the dried material. After drying in the rotary evaporator, the sample was dried overnight at 120° C. The catalyst was finally calcined at 300° C. in flowing air for 5 hours with a ramp rate of 2° C./min.

Known methods can be applied to predict whether a stable structure can be achieved with a given combination of cations.

The resultant materials were studied using characterization techniques including X-ray diffraction, electron microscopy, elemental analysis and X-ray absorption spectroscopy.

### Experimental Results

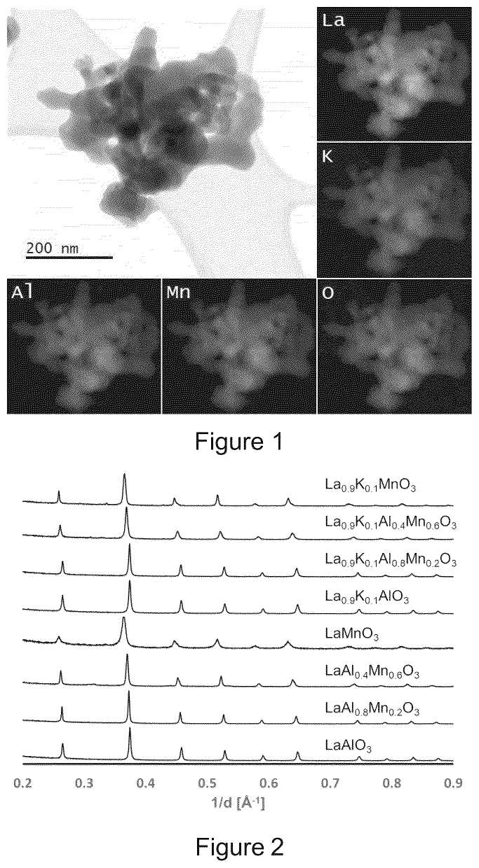

FIGS. 1 and 2 are pertinent to the following discussion. For convenience, the embodiments of the disclosed catalyst support structures used in the experiments are referred to as “modified” materials. The results of all experiments conducted showed that replacement or displacement of La and Al (La₍₁₋ₓ₎KₓAl_{(1-y)}Mn_{y}O₃ wherein x≤0.1 and y≤1) resulted in the formation of a perovskite structure with K located on the A site and Mn located on the B site. Negligible agglomeration of the K or Mn species was observed on the surfaces of the modified support structures.

Moreover, the disclosed materials were stable upon exposure to reducing conditions at elevated temperatures, suggesting that no exsolution was taking place.

Surface area of the modified support structures can optionally be increased by modifying the synthesis approach. Thus, the perovskite phase can be grown in the presence of poly(methyl methacrylate) (PMMA) microspheres which are subsequently removed by calcination. This approach can be used to produce the modified perovskite support structures with high purity and stability, and with increased specific surface area comparable with that of classic support materials. This further facilitates the deposition of the active phase via classic impregnation techniques.

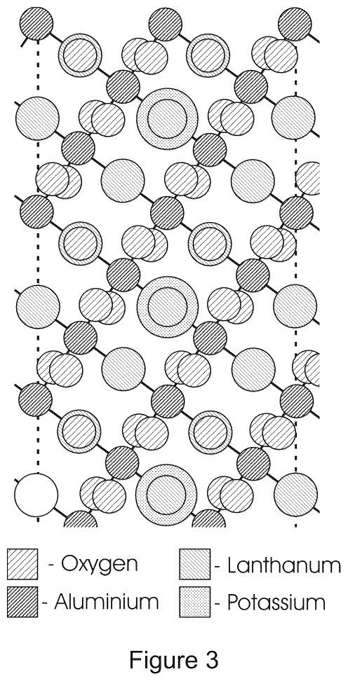

FIG. 3 is presented only as an illustration of one example of a catalyst support structure as disclosed herein. It will be appreciated that the scope of the invention is not limited to the illustrated embodiment nor even to perovskites or other multi-metal oxides.

FIG. 3 provides a schematic illustration of a lattice structure of a modified perovskite catalyst support structure as disclosed herein (La_{0.9}K_{0.1}AlO₃), showing how atoms of a promoter (K in this case) can be anchored or locked into the support structure so that they have fixed positions relative to the atoms of the framework structure. The figure shows several unit cells of a slab model generated for the support structure. The La, Al and O atoms can be considered as a first group of atoms bonded to one another to provide a structural framework for supporting the catalyst, and the K atoms can be considered as a second group of atoms comprising promoter atoms dispersed within the structural framework and bonded to the first group of atoms such that the promoter atoms are anchored or fixed in position relatively to the first group of atoms. The catalyst is not shown in the drawing.

### Comparative Results

Pre-prepared Fe₂O₃ nanoparticles (average crystallite size approximately 20 nm; Fe₂O₃ loading of 20 wt. %; nomenclature: Fe-La₍₁₋ₓ₎KₓAl_{(1-y)}Mn_{y}O₃) were deposited onto the modified and unmodified perovskite supports as well as onto conventional supports (ZrO₂, Al₂O₃, SiO₂, TiO₂). These materials were tested under Fischer-Tropsch conditions in a slurry reactor at 240° C., 15 bar and a H₂ to CO ratio of 2. To compare the effect of potassium incorporated (locked) into the perovskite structure to conventional potassium promotion, a Fe—LaAlO₃ sample was impregnated with 0.85 wt. % K (nomenclature: Fe-05K/LaAlO₃). This equated to half of the absolute amount of potassium in Fe—La_{0.9}K_{0.1}AlO₃ although the impregnated potassium was only located on the surface of the LaAlO₃ while the weight percentage of potassium on the surface in the Fe—La_{0.9}K_{0.1}AlO₃ was much lower as it was evenly distributed throughout the perovskite phase.

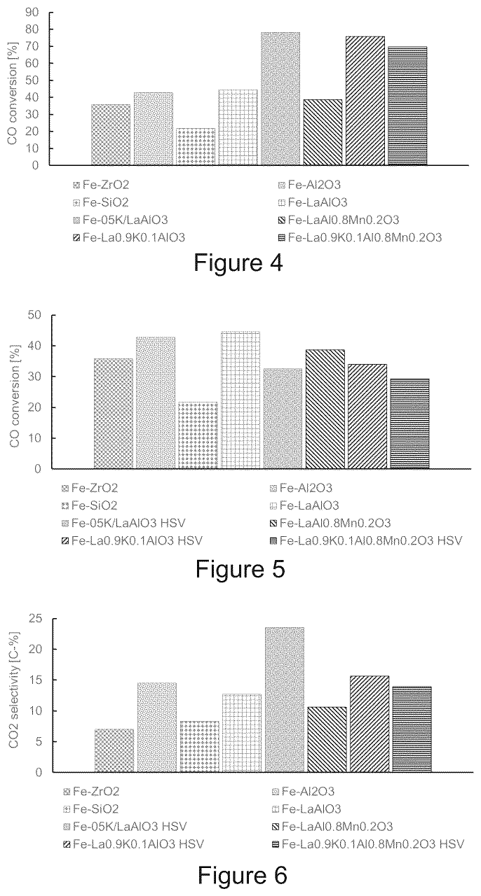

FIGS. 4 and 5 illustrates how, under the chosen conditions, iron supported on conventional supports can display CO conversions between about 20% and 45%. The unmodified Fe—LaAlO₃ matched the 45% conversion. At the lowest concentrations of Mn replacing Al in the modified materials, the conversion was only reduced slightly to just under 40%. When K was incorporated (locked) into the perovskite structure (Fe—La_{0.9}K_{0.1}AlO₃) a significant increase in activity was observed, up to about 76% CO conversion. In the presence of Mn (Fe—La_{0.9}K_{0.1}Al_{0.8}Mn_{0.2}O₃) the conversion remained elevated (70%). Addition of 0.85 wt. % potassium via impregnation of Fe—LaAlO₃ also increased the the conversion, to 78%.

To compare catalyst performances the space velocity (SV—gas flow per g of catalyst) was increased by a factor of 3.75 for the samples with enhanced activity (samples with suffix HSV in FIG. 5). FIGS. 4 and 5 illustrate that all samples have a similar conversion allowing for a comparison of selectivities.

FIG. 6 is now discussed. An undesired product in the Fischer-Tropsch synthesis is CO₂, formed via the water gas shift reaction of CO and water. Therefore, lower CO₂ selectivity is an advantage. However, promotion with Mn and K increases the binding strength of CO onto the surface of the catalyst and therefore increases water gas shift activity of the iron catalyst, and therefore the CO₂ selectivity. FIG. 6 illustrates an example of this behaviour. When K is added to Fe—LaAlO₃ via impregnation, the CO₂ selectivity increased from 13 C-% to 24 C-%.

By contrast, although locking of K and/or Mn into the modified perovskite structures increased CO conversion necessitating a higher SV compared against Fe—LaAlO₃, it did not enhance CO₂ selectivity to the same extent as in the case of Fe-05K/LaAlO₃. The presence of both K and Mn only increased the CO₂ selectivity by 1 C-% at a much higher rate of CO conversion.

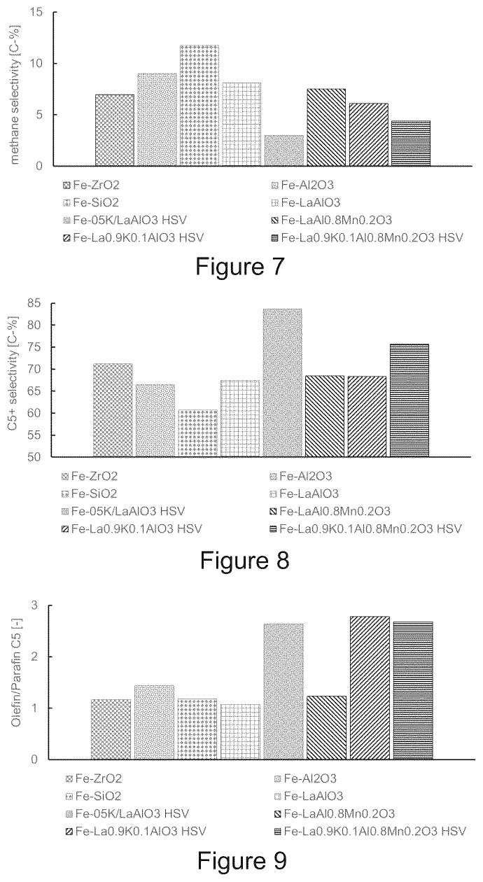

FIG. 7 is now discussed. Methane (CH₄) is a further undesired product in FTS. In this regard, the incorporation of Mn has a slightly positive effect on CH₄ selectivity, while K supresses the full hydrogenation, i.e., methane formation. Embodiments having potassium incorporated into the perovskite structure were outperformed in this regard by the impregnated potassium. However, CH₄ selectivity for all promoter containing samples was below 7.5 C-% and lower than for Fe—LaAlO₃, as is illustrated in FIG. 7.

FIG. 8 illustrates the selectivity to longer hydrocarbons, namely of a chain length of 5 carbons and above. Compared to Fe—LaAlO₃ the selectivity to longer hydrocarbons remains stable upon incorporation of K into the perovskite matrix while at a higher CO conversion rate. In the presence of K and Mn in the perovskite, the C5+ selectivity was increased by 9 C-%.

FIG. 9 illustrates the olefin to paraffin ratio of the C5 hydrocarbon fraction. The C5 fraction was chosen as representative fraction of the hydrocarbon product. The incorporation of K into the perovskite enhanced the olefin to paraffin fraction from 1.1 to 2.8. In the presence of K and Mn this ratio was 2.7.

Iron nitrate was impregnated via incipient wetness impregnation onto the La₁₋ₓKₓAlO₃—PMMA (x=0 and 0.1) perovskites to achieve an iron loading of 16 wt.-%. These materials were tested under Fischer-Tropsch conditions in a slurry reactor at 240° C., 15 bar and a H₂ to CO ratio of 2.

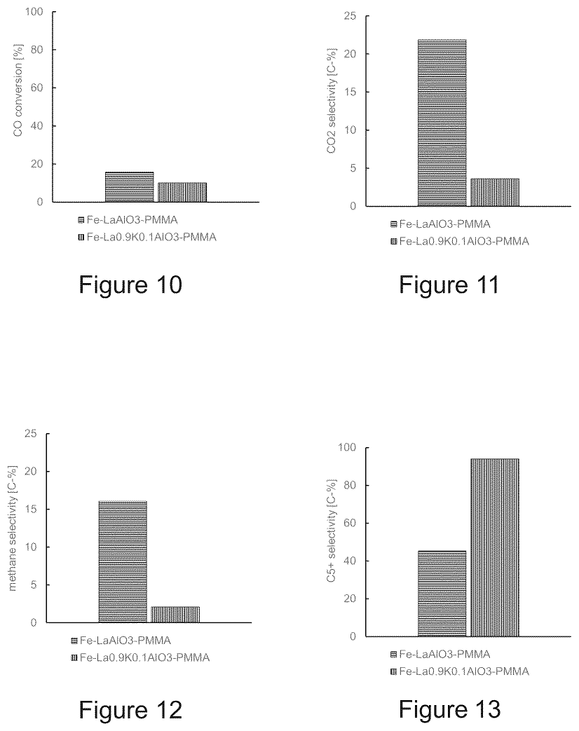

FIG. 10 illustrates the CO conversion obtained for both samples and shows that the conversion is slightly lower in the presence of K in the perovskite structure.

FIG. 11 shows that K incorporation surprisingly reduces the CO₂ selectivity from 22 to 4 C-%.

FIG. 12 illustrates that in parallel to the CO₂ selectivity, the methane selectivity, another undesired product of the FTS, also dropped from 16 to 2 C-%.

FIG. 13 illustrates how in turn the selectivity to long chained hydrocarbons increased to 94 C-% in the presence of K.

It will be appreciated that the FTS process was selected for the experiments only as an example for illustrative purposes. As previously mentioned, the modified perovskites and many other embodiments of the disclosed catalyst support structures may be expected to be useful in numerous other catalytic reactions.

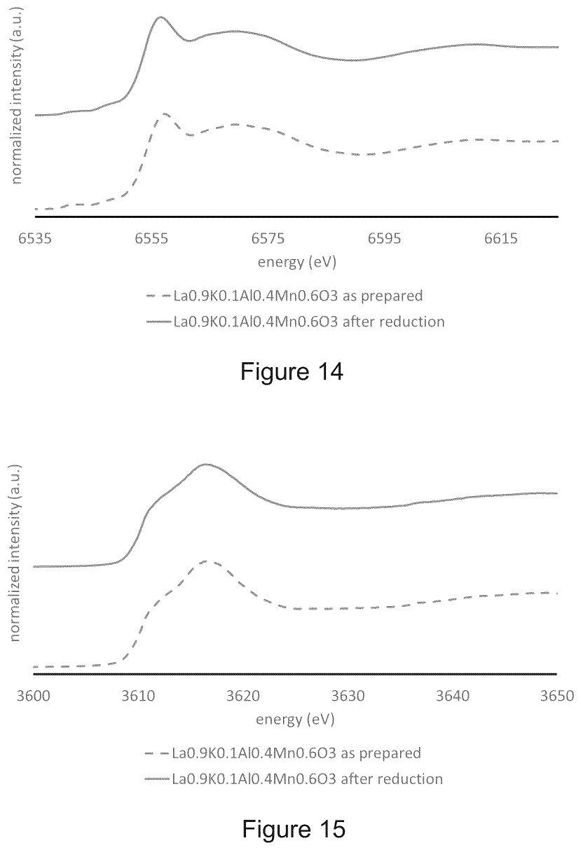

FIGS. 14 and 15 illustrate exemplary catalytic reaction conditions under which embodiments of the disclosed catalyst support structure have been shown to be stable. The reaction conditions in this instance include reductive treatment in hydrogen at 450° C. It will be appreciated, however, that numerous other sets of reaction conditions will apply in other catalytic reactions for which the disclosed support structures may be suitable.

The disclosed catalyst support structures may provide advantages over classical support structures such as those impregnated with promoters by standard methods. These advantages may include the following:
Enhanced catalyst performance. In the examples provided, catalysts supported on the structures described herein outperformed classic catalysts in iron-based Fischer-Tropsch synthesis, both with regards to activity and selectivity.
The support structures provide a separating functionality combined with a promoting functionality. The disclosed support structures offer functionality to separate catalyst materials in the same way that conventional support structures do; however, they go further by also providing a promoting functionality within their structures. This may permit better control of contact between catalyst and promoter.
Inhibition or suppression of promoter movement or migration within the support structures. Since the promoter element or elements are incorporated into the crystal lattice (and therefore locked or positionally anchored in the structure), movement of the atoms of these elements within the structures may be reduced. This may inhibit sintering and the associated loss of active surface area, even in harsh reaction conditions. It may also potentially open new (higher) promoter concentration windows that have not previously been practical.
The “locking in” or immobilization of the promoters can provide a more regular or uniform distribution of atoms of the promoter across the surface of the support.
Inhibition of phase change of promoters.
Surface structure improvements.
Less agglomeration of promoter elements since the elements are atomically dispersed and distributed more widely and evenly across and within the support structure than in classical supports.
Increased stability of the disclosed support structures compared to conventional support structures, when exposed to reducing conditions at elevated temperatures. Exsolution is typically negligible.
Increased stability of the promoter elements by comparison with conventional promotion e.g., by comparison with conventional K promotion with Fe—K/LaAlO₃.
Speciation and location of the promoter elements may be better defined, and these factors may be less likely to change under reaction conditions.
Specifically with regard to FTS processes, use of the disclosed support structures may cause a comparative decrease in undesirable CO₂ selectivity, a result which would have been entirely unexpected by those skilled in the art.
For some embodiments, an elevated olefinicity in the hydrocarbon product when co-promoters K and Mn are employed, e.g., when using Fe—La_{0.9}K_{0.1}Al_{0.8}Mn_{0.2}O₃.
The support structures may permit the deposition of the active phase(s) of a catalyst via classic impregnation techniques, particularly although not exclusively if the support structures are grown in the presence of templates such as PMMA spheres.

Incorporation and locking of promoter elements into complex inorganic oxide structures and the use of the resulting structures as catalyst supports has not previously been described. In past approaches, perovskites were used as precursors to catalysis and it was intended that the catalytically active metal should exsolute during the reaction. The aim was to provide perovskites that are unstable, and which would potentially be destroyed during the catalyst formation process. By contrast, the presently disclosed support structures are not intended to be destroyed and are synthesized with a view to providing a stable structure rather than a destabilized structure. As a result, the promoters generally do not exsolute under reaction conditions. That is a key difference between the present support structures and all others which have preceded them. For the first time the promoter elements of a heterogenous catalyst reaction are locked in atomic distribution inside the perovskite support structure (not added to the outside surface) and still achieve their promoting effect.

A further distinction is that classical catalyst support structures are intended to operate primarily as platforms or substrates to support or carry the promoters. Their purpose is to act as a physical separator for separating different regions of the promoter elements.

Possible reasons for the advantageous effects of the disclosed supports may include better distribution of the promoters and a resulting better contact with the catalytic phase. The promoter elements, e.g., K and Mn, may be atomically dispersed across and within the structure of the support material, which may have advantageous effects with regards to activity and selectivity.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the accompanying claims.

As used herein, the word “promoter” will be understood to mean a substance for use in combination with a solid catalyst to improve its performance in a chemical reaction. Without limiting the generality thereof, the improvements in performance conferred by a promoter (or promoter element) may arise from one or more mechanisms such as increases in the activity of the catalyst, increases in available surface area, stabilization of the catalyst (e.g., stabilization against crystal growth and sintering), or improvements in mechanical strength. The promoter need not have any catalytic properties of its own. Some non-limiting examples of promoters, presented for illustrative purposes only, include K, Mn and Cu for the FTS process, K for the Haber-Bosch process, and chromic oxide for the manufacture of methyl alcohol from water gas.

The phrase “promoter element” will be understood to mean a chemical element which is effective to function as a promoter of a catalytic reaction, whether present in elemental form or as part of a molecule or crystalline substance comprising other elements.

Without limiting the generality thereof, the word “exsolution” and variations such as “exsolute” or “exsoluted” will be interpreted to refer to exsolution of one or more elements from the crystal lattice of the catalyst support structure.

Without limiting the generality thereof, the term “stable against collapse and exsolution” will be interpreted to mean that the following characteristics of the relevant catalyst support structures are negligible for practical purposes during use:
collapse or structural degradation of the relevant catalyst support structure; and
exsolution of one or more elements from the crystal lattice.

These properties of stability provide critical features and advantages of the presently disclosed support structures and their use, distinguishing them over previous descriptions of perovskites used for heterogeneous catalysis.

Phrases connoting the concept of supporting or carrying a catalyst shall have their widest meaning. Without limitation thereto, the word “supporting” and variations thereof may refer to any of the following actions amongst others: chemical bonding (including ionic bonding) between the active species of the catalyst and the support structure, attraction by intermolecular forces such as Van der Waals forces, adsorption onto, absorption by, or impregnation of the active species into the disclosed catalyst support structures.

Finally, throughout the specification and accompanying claims, unless the context requires otherwise, the word “comprise” or variations such as “comprises” or “comprising” will be understood to imply the inclusion of a stated integer or group of integers but not the exclusion of any other integer or group of integers.

### REFERENCE

Goldwasser, M. R. et al. Modified iron perovskites as catalysts precursors for the conversion of syngas to low molecular weight alkenes. J. Mol. Catal. A: Chem. 193, 227-236, doi:https://doi.org/10.1016/S1381-1169(02)00472-7 (2003).

1. A method of preparing a catalyst support structure for use in a catalytic reaction, the method comprising the steps of:
synthesizing a mixed metal oxide compound having a crystallographic phase which defines a crystal lattice, the compound being configured to support a catalyst for a catalytic reaction, the compound further having a group of cations of at least one catalytic promoter element being dispersed within the compound and incorporated into the crystal lattice, and the promoter element being capable of promoting the catalytic reaction;
wherein the conditions of the synthesis are preselected to inhibit destabilization of the catalyst support structure such that the structure remains stable against collapse and exsolution under reaction conditions associated with the catalytic reaction.

2. The method according to claim 1, wherein the conditions of the synthesis are preselected such that the metal oxide compound is synthesized as an oxidic perovskite having the following formula CHEM 1:
A₍₁₋ₓ₎A′₍ₓ₎B_{(1-y)}B′_{y}O₃ CHEM 1:
wherein:
A and B represent metal cations having ionic radii R_{A} and R_{B} respectively;
A′ and B′ represent cations of at least one promoter element;
O represents an oxygen atom having an ionic radius R_{O}; and
R_{A}+R_{O}=t×sqrt(R_{B}+R_{O}), wherein t has a value ranging from about 0.7 to about 1.3.

3. The method according to claim 2, wherein the metal cation A comprises a cation of La or Bi, the metal cation B comprises a cation of an element selected from the group consisting of Al, Ti, Zn and Mo, and the (or each) promoter element is independently selected from the group consisting of alkali, alkaline earth, and transition metals of groups 3 to 7 and periods 4 to 5 of the periodic table.

4. The method according to claim 2, wherein the perovskite comprises La₍₁₋ₓ₎KₓAl_{(1-y)}Mn_{y}O₃ wherein 0<x≤0.2 and y≤1.

5. The method according to a claim 1, which further includes a step of depositing onto the metal oxide compound a catalyst capable of catalysing said catalytic reaction, this step being performed subsequently to the step of synthesizing the metal oxide compound.

6. The method according to claim 1, wherein the catalystic reaction is a reaction selected from the group consisting of Fischer-Tropsch syntheses, Haber-Bosch processes, decomposition of nitrogen oxides (NOx) and N₂O, dry reforming of CO₂, steam reforming of methane, CO and CO₂ hydrogenation, (reverse) water gas shift reactions, soot oxidation, and synthesis of higher alcohols over Cu based catalysts.

7. The method according to claim 1, wherein the reaction conditions are selected from groups of conditions suitable for Fischer-Tropsch synthesis, Haber-Bosch processes, decomposition of nitrogen oxides (NOx) and N₂O, dry reforming of CO₂, steam reforming of methane, CO and CO₂ hydrogenation, (reverse) water gas shift reactions, soot oxidation, synthesis of higher alcohols, and synthesis over Cu based catalysts.

8. A catalyst support structure prepared using the method according to claim 1.

9. A catalyst support structure comprising
a mixed metal oxide compound having a crystallographic phase which defines a crystal lattice, the compound being configured to support a catalyst for a catalytic reaction; and
a group of cations of at least one catalytic promoter element dispersed within the compound and incorporated into the crystal lattice, the promoter element being capable of promoting the catalytic reaction;
wherein the catalyst support structure is stable against collapse and exsolution under reaction conditions associated with the catalytic reaction.

10. A catalyst support structure according to claim 9, wherein the mixed metal oxide compound comprises an oxidic perovskite having the following formula CHEM 1:
A₍₁₋ₓ₎A′₍ₓ₎B_{(1-y)}B′_{y}O₃ CHEM 1:
wherein:
A and B represent metal cations having ionic radii R_{A} and R_{B} respectively;
A′ and B′ represent cations of at least one promoter element;
O represents an oxygen atom having an ionic radius R_{O}; and
R_{A}+R_{O}=t×sqrt(R_{B}+R_{O}), wherein t has a value ranging from about 0.7 to about 1.3.

11. The catalyst support structure according to claim 10, wherein the metal cation A comprises a cation of La or Bi, the metal cation B comprises a cation of an element selected from the group consisting of Al, Ti, Zn and Mo, and the (or each) promoter element is independently selected from the group consisting of alkali, alkaline earth, and transition metals of groups 3 to 7 and periods 4 to 5 of the periodic table.

12. The catalyst support structure according to claim 10, wherein the perovskite comprises La₍₁₋ₓ₎KₓAl_{(1-y)}Mn_{y}O₃ wherein 0<x≤0.2 and y≤1.

13. A catalyst support structure for use in heterogeneous catalysis of a chemical reaction, the structure comprising a perovskite with a crystal lattice defining a surface configured to support an active catalyst phase and having a plurality of atoms of at least one promoter element distributed generally uniformly across the surface and within the crystal lattice; wherein said promoter element is effective to promote catalysis of the chemical reaction.

14. An assembly comprising a catalytically active phase and a catalyst support structure according to claim 9 onto which the catalytically active phase is loaded, wherein the catalytically active phase is substantially free of cations of the promoter element.

15. A method of performing a catalytic reaction, the method comprising the steps of supporting a catalyst on a catalyst support structure according to claim 9, subjecting the catalyst to activation treatment thereby to provide a catalytically active phase of the catalyst loaded on the catalyst support structure, exposing the catalyst support structure with the loaded active phase to reaction conditions, contacting at least one reactant with the loaded active phase, and applying activation energy to the reactant, thereby to convert the reactant to at least one product.
